(19) United States
(12) Patent Application Publication
(10) Pub. No.: US 2008/0214572 A1
(43) Pub. Date: Sep. 4, 2008

(54) Selected Tetracyclic Tetrahydrofuran Derivatives Containing a Cyclic Amine Side Chain

(76) Inventors: Francisco Javier Fernandez-Gadea, Toledo (ES); Jose Ignacio Andres-Gil, Madrid (ES); Andres Avelino Trabanco-Suarez, Olias del Rey (Toledo) (ES); Jose Maria Cid-Nunez, Toledo (ES); Antonius Adrianus Hendrikus Petrus Megens, Beerse (BE)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/914,169

(22) PCT Filed: May 17, 2006
(86) PCT No.: PCT/EP06/62362

§ 371 Date: Nov. 12, 2007

(30) Foreign Application Priority Data
May 19, 2005 (EP) 05104279.4

(51) Int. Cl. A61K 31/495; C07D 241/04; C07D 211/14; A61K 31/4453; A61P 25/00; A61P 25/18; A61P 25/06

(52) U.S. Cl. 514/255.04; 544/380; 546/195; 514/319

(57) ABSTRACT

This invention concerns novel tetracyclic tetrahydrofuran derivatives containing a cyclic amine side chain with binding affinities towards serotonin receptors, in particular 5-HT_{2A} and 5-HT_{2C} receptors, and towards dopamine receptors, in particular dopamine D₂ receptors, pharmaceutical compositions comprising the compounds according to the invention, the use thereof as a medicine, in particular for the prevention and/or treatment of a range of psychiatric and neurological disorders, in particular certain psychotic, cardiovascular and gastrokinetic disorders and processes for their production.

The compounds according to the invention can be represented by general Formula (I)

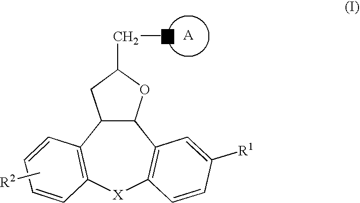

and comprises also a pharmaceutically acceptable acid or base addition salt thereof, a stereochemically isomeric form thereof, an N-oxide form thereof, and a quaternary ammonium salt thereof, wherein all substituents are defined as in claim 1.

### FIELD OF THE INVENTION

This invention concerns novel substituted tetracyclic tetrahydrofuran derivatives containing a cyclic amine side chain with binding affinities towards serotonin receptors, in particular 5-HT_{2A} and 5-HT_{2C} receptors, and towards dopamine receptors, in particular dopamine D₂ receptors, pharmaceutical compositions comprising the compounds according to the invention, the use thereof as a medicine, in particular for the prevention and/or treatment of a range of psychiatric and neurological disorders, in particular certain psychotic, cardiovascular and gastrokinetic disorders and processes for their production.

### BACKGROUND PRIOR ART

WO 97/38991, published Oct. 23, 1997 (Janssen Pharmaceutica N.V.) discloses substituted tetracyclic tetrahydrofuran derivatives that may be used as therapeutic agents in the treatment or prevention of CNS disorders, cardiovascular disorders or gastrointestinal disorders. In particular, the compounds show affinity for the serotonin 5-HT₂ receptors, particularly for the 5-HT_{2A} and 5-HT_{2C} -receptors.

WO 99/19317, published Apr. 22, 1999 (Janssen Pharmaceutica N.V.) discloses substituted tetracyclic tetrahydrofuran derivatives with a specific halogen substitution pattern on the dibenzoazepine, dibenzooxepine, dibenzothiepine or dibenzosuberane ring. The compounds are useful in the treatment or prevention of CNS disorders, cardiovascular disorders or gastrointestinal disorders and show a faster onset of action over the compounds as disclosed in WO 97/38991.

Both WO 03/048146, published Jun. 12, 2003 (Janssen Pharmaceutica N.V.) and WO 03/048147, published Jun. 12, 2003 (Janssen Pharmaceutica N.V.) disclose processes for the preparation of each of the four diastereoisomers of trans-, respectively cis-fused 3,3a,8,12b-tetrahydro-2H-dibenzo[3,4:6,7]cyclohepta[1,2-b]furan derivatives in a stereochemically pure form from a single enantiomerically pure precursor. The compounds of WO 03/048146 show affinity for 5-HT₂ receptors, particularly for 5-HT_{2A} and 5-HT_{2C} receptors. The compounds of WO 03/048147 show affinity for the serotonin 5-HT_{2A}, 5-HT_{2C} and 5-HT₇ receptors, the H₁-receptors (pIC₅₀=7.15-7.89), D2 and/or D3 receptors and for the norepinephrine reuptake transporters (pIC₅₀=6.03-7.34). The compounds disclosed in the latter two publications do not contain a cyclic amine side chain.

WO 03/040122, published May 15, 2003 (Janssen Pharmaceutica N.V.) discloses mandelate salts of the compounds according to WO 97/38991 and WO 99/19317. Said salts were surprisingly found to be more stable at enhanced temperature and relative humidity than the compounds disclosed in WO 97/38991 and WO 99/19317.

### DESCRIPTION OF THE INVENTION

It is the object of the present invention to provide novel analogues of the tetracyclic tetrahydrofuran derivatives of WO-publications WO 97/38991 and WO 99/19317 which have an advantageous pharmacological profile in comparison with the compounds disclosed in said WO-publications.

This goal is achieved by the present novel compounds according to Formula (I):

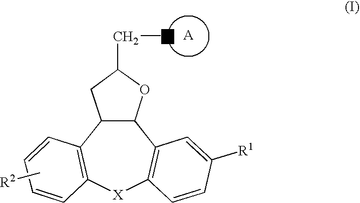

a pharmaceutically acceptable acid or base addition salt thereof, a stereochemically isomeric form thereof, an N-oxide form thereof, and a quaternary ammonium salt thereof, wherein:
R¹ is hydrogen, halo or C₁₋₆alkyloxy;
R² is hydrogen or cyano; and
a) X is O or S; and

A is a radical of formula (a-1), (a-2) or (a-3),

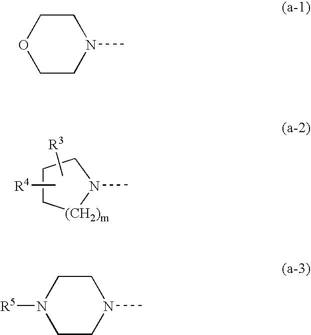

wherein:
m is an integer equal to zero, 1, 2 or 3;
R³ and R⁴ are each independently hydrogen, C₁₋₆alkyl or aryl; and
R⁵ is hydrogen; C₁₋₆alkyl; C₁₋₆alkylcarbonyl; C₁₋₆alkyl-carbonyloxyalkyl; C₁₋₆alkyloxycarbonyl; aryl; or C₁₋₆alkyl substituted with one or more substituents selected from hydroxy, C₁₋₆alkyloxy, C₁₋₆alkylcarbonyloxy and aryl; or
b) X is CH₂; and

A is a radical of formula (a-2) or (a-3) above wherein:
m is an integer equal to zero, 1, 2 or 3;
R³ and R⁴ are each independently hydrogen or C₁₋₆alkyl; and
R⁵ is hydrogen; C₂₋₆alkyl; C₁₋₆alkylcarbonyl C₁₋₆alkylcarbonyloxyalkyl; C₁₋₆alkyloxycarbonyl; or C₁₋₆alkyl substituted with one or more substituents selected from hydroxy and aryl, with the proviso that 2-hydroxyethyl is excluded; and
aryl is phenyl; or phenyl substituted with 1, 2 or 3 substituents selected from halo, hydroxy, C₁₋₆alkyl and halomethyl.

The compounds according to the invention are structurally characterized by the presence of a cyclic amine side chain in the 2-position. It has been found that the presence of this side chain provides compounds which have a potent affinity for the D₂ receptor, an activity not attributed to the compounds in the above-mentioned WO applications WO 97/38991 and WO 99/19317, which renders the compounds according to the invention especially suitable for use in the treatment of psychoses such as mania, excitement, aggression, and the positive symptoms of schizophrenia. In contrast, the compounds according to the invention do not show any significant inhibitory activity against norepinephrine transporter reuptake (NET), which indicates that they do not have a useful antidepressant activity. The absence of such antidepressant activity may be advantageous when selecting a compound for a certain therapeutic profile, particularly since the compounds further have affinity towards the 5-HT_{2A} and 5-HT_{2C} receptors. Such a profile of activity for the compounds according to the invention is not taught or suggested in the above WO publications.

More in particular, the invention relates to a compound according to the invention of general Formula (I), a pharmaceutically acceptable acid or base addition salt thereof, a stereochemically isomeric form thereof, an N-oxide form thereof, and a quaternary ammonium salt thereof, wherein:
R¹ is halo;
R² is hydrogen;
aryl is phenyl; or phenyl substituted with halo or halomethyl.

More in particular, the invention relates to a compound according to the general Formula (I), a pharmaceutically acceptable acid or base addition salt thereof, a stereo-chemically isomeric form thereof, an N-oxide form thereof, and a quaternary ammonium salt thereof, wherein:
X is O or S;
A is a radical of formula (a-1), (a-2) or (a-3) above, wherein
   m is an integer equal to 1 or 2;
   R³ and R⁴ are each independently hydrogen or aryl; and
   R⁵ is C₁₋₆alkyl or C₁₋₆alkyl substituted with an hydroxy substituent.

Particularly preferred compounds according to the invention include the base compounds corresponding to Nos. 5, 12, 17, 27, 28, 29, 34, 35, 36, 39, 44 and 46, identified in the application, in particular in Table 1 below, and a pharmaceutically acceptable acid or base addition salt thereof, a stereochemically isomeric form thereof, an N-oxide form thereof, and a quaternary ammonium salt thereof. More particularly, it concerns compounds according to one of the following structural formulas (I-1) to (1-12) depicted below, a pharmaceutically acceptable acid or base addition salt thereof, a stereochemically isomeric form thereof, an N-oxide form thereof, and a quaternary ammonium salt thereof:

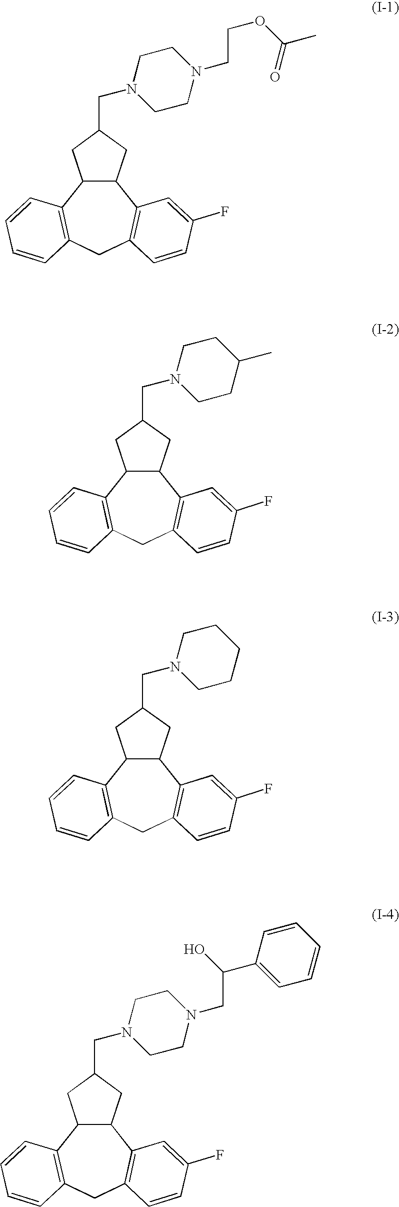

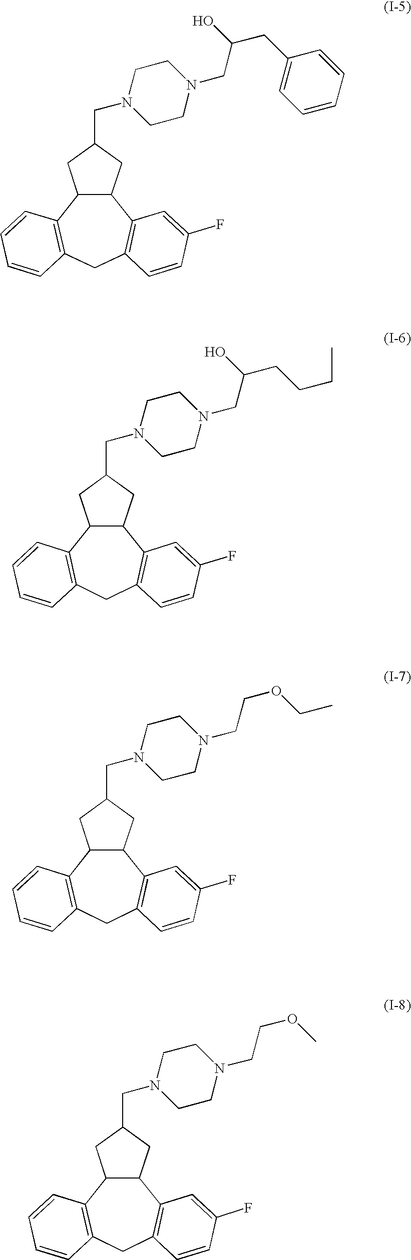

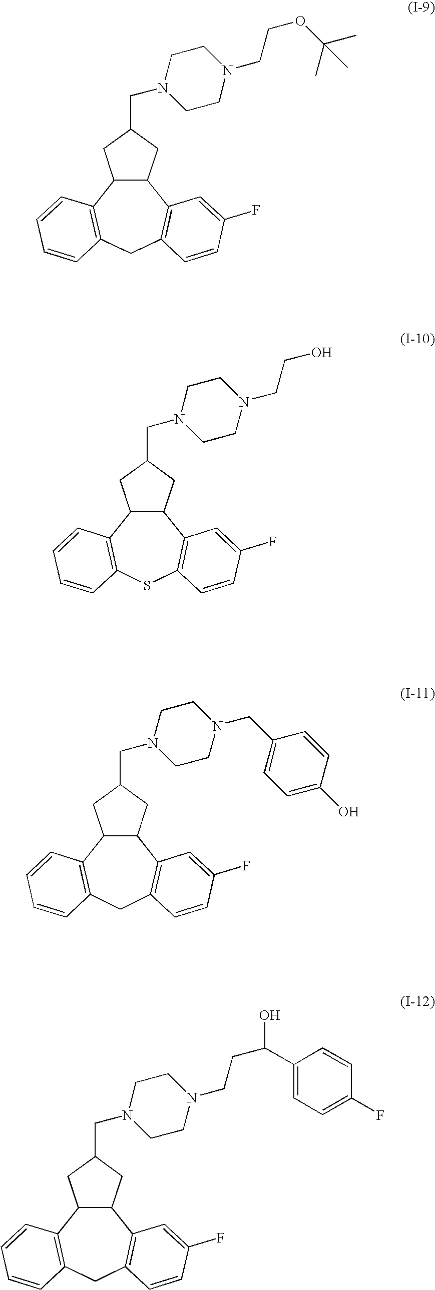

### DETAILED DESCRIPTION OF THE INVENTION

In the framework of this application, alkyl is defined as a monovalent straight or branched saturated hydrocarbon radical having from 1 to 6 carbon atoms, or if indicated otherwise, from 2 to 6 carbon atoms, for example methyl, ethyl, propyl, butyl, 1-methylpropyl, 1,1-dimethylethyl, pentyl, hexyl; alkyl further defines a monovalent cyclic saturated hydrocarbon radical having from 3 to 6 carbon atoms, for example cyclopropyl, methylcyclopropyl, cyclobutyl, cyclopentyl and cyclohexyl. The definition of alkyl also comprises, unless otherwise specified, an alkyl radical that is optionally substituted on one or more carbon atoms with one or more phenyl, halo, cyano, oxo, hydroxy, formyl and amino radicals, for example hydroxyalkyl, in particular hydroxymethyl and hydroxyethyl and polyhaloalkyl, in particular difluoromethyl and trifluoromethyl.

In the framework of this application, halo is generic to fluoro, chloro, bromo and iodo.

In the framework of this application, with “compound(s) according to the invention” is meant a compound according to the general Formula (I), a pharmaceutically acceptable acid or base addition salt thereof, a stereochemically isomeric form thereof, an N-oxide form thereof, and a quaternary ammonium salt thereof.

The pharmaceutically acceptable salts are defined to comprise the therapeutically active non-toxic acid addition salts forms that the compounds according to Formula (I) are able to form. Said salts can be obtained by treating the base form of the compounds according to Formula (I) with appropriate acids, for example inorganic acids, for example hydrohalic acid, in particular hydrochloric acid, hydrobromic acid, sulfuric acid, nitric acid and phosphoric acid; organic acids, for example acetic acid, hydroxyacetic acid, trifluoroacetic acid, propanoic acid, lactic acid, pyruvic acid, oxalic acid, malonic acid, succinic acid, maleic acid, mandelic acid, fumaric acid, malic acid, tartaric acid, citric acid, methanesulfonic acid, ethanesulfonic acid, benzenesulfonic acid, p-toluenesulfonic acid, cyclamic acid, salicylic acid, p-aminosalicylic acid, pamoic acid and mandelic acid. Preferred salts are obtained from trifluoroacetic acid (.C₂HF₃O_{2,}, trifluoroacetate), oxalic acid (.C₂H₂O₂, oxalate) and mandelic acid (.C₆H₅C₂O₃H₃, mandelate).

Conversely, said salts forms can be converted into the free forms by treatment with an appropriate base.

The term addition salt as used in the framework of this application also comprises the solvates that the compounds according to Formula (I) as well as the salts thereof, are able to form. Such solvates are, for example, hydrates and alcoholates.

The N-oxide forms of the compounds according to Formula (I) are meant to comprise those compounds of Formula (I) wherein one or several nitrogen atoms are oxidized to the so-called N-oxide, particularly those N-oxides wherein one or more tertiary nitrogens (e.g. particularly those tertiary nitrogens bearing the R¹ and R² substituents) are N-oxidized. Such N-oxides can easily be obtained by a skilled person without any inventive skills and they are obvious alternatives for the compounds according to Formula (I) since these compounds are metabolites, which are formed by oxidation in the human body upon uptake. As is generally known, oxidation is normally the first step involved in drug metabolism (Textbook of Organic Medicinal and Pharmaceutical Chemistry, 1977, pages 70-75). As is also generally known, the metabolite form of a compound can also be administered to a human instead of the compound per se, with much the same effects.

The compounds according to the invention possess at least one oxidizable nitrogen (tertiary amine moiety). It is therefore highly likely that N-oxides are to form in the human metabolism.

The compounds of Formula (I) may be converted to the corresponding N-oxide forms following art-known procedures for converting a trivalent nitrogen into its N-oxide form. Said N-oxidation reaction may generally be carried out by reacting the starting material of Formula (I) with an appropriate organic or inorganic peroxide. Appropriate inorganic peroxides comprise, for example, hydrogen peroxide, alkali metal or earth alkaline metal peroxides, e.g. sodium peroxide, potassium peroxide; appropriate organic peroxides may comprise peroxy acids such as, for example, benzenecarboperoxoic acid or halo substituted benzenecarboperoxoic acid, e.g. 3-chloro-benzenecarboperoxoic acid, peroxoalkanoic acids, e.g. peroxoacetic acid, alkylhydro-peroxides, e.g. tert-butyl hydroperoxide. Suitable solvents are, for example, water, lower alkanols, e.g. ethanol and the like, hydrocarbons, e.g. toluene, ketones, e.g. 2-butanone, halogenated hydrocarbons, e.g. dichloromethane, and mixtures of such solvents.

A quaternary ammonium salt of compound according to Formula (I) defines said compound which is able to form by a reaction between a basic nitrogen of a compound according to Formula (I) and an appropriate quaternizing agent, such as, for example, an optionally substituted alkylhalide, arylhalide or arylalkylhalide, in particular methyliodide and benzyliodide. Other reactants with good leaving groups may also be used, such as, for example, alkyl trifluoromethanesulfonates, alkyl methanesulfonates and alkyl p-toluenesulfonates. A quaternary ammonium salt has at least one positively charged nitrogen. Pharmaceutically acceptable counterions include chloro, bromo, iodo, trifluoroacetate and acetate ions.

The invention also comprises a derivative compound (usually called “pro-drug”) of a pharmacologically-active compound according to the invention, in particular according to Formula (1), which is degraded in vivo to yield a compound according to the invention. Pro-drugs are usually (but not always) of lower potency at the target receptor than the compounds to which they are degraded. Pro-drugs are particularly useful when the desired compound has chemical or physical properties that make its administration difficult or inefficient. For example, the desired compound may be only poorly soluble, it may be poorly transported across the mucosal epithelium, or it may have an undesirably short plasma half-life. Further discussion on pro-drugs may be found in Stella, V. J. et al., “Prodrugs”, Drug Delivery Systems, 1985, pp. 112-176, and Drugs, 1985, 29, pp. 455-473.

A pro-drug form of a pharmacologically-active compound according to the invention will generally be a compound according to Formula (I), a pharmaceutically acceptable acid or base addition salt thereof, an N-oxide form thereof, or a quaternary ammonium salt thereof, having an acid group which is esterified or amidated. Included in such esterified acid groups are groups of the formula —COOR^{x}, where R^{x} is a C₁₋₆alkyl, phenyl, benzyl or one of the following groups:

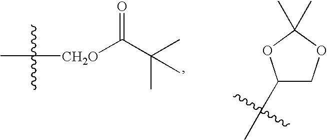

Amidated groups include groups of the formula —CONR^{y}R^{z}, wherein R^{y} is H, C₁₋₆alkyl, phenyl or benzyl and R^{z} is —OH, H, C₁₋₆alkyl, phenyl or benzyl. A compound according to the invention having an amino group may be derivatised with a ketone or an aldehyde such as formaldehyde to form a Mannich base. This base will hydrolyze with first order kinetics in aqueous solution.

The term “stereochemically isomeric forms” as used hereinbefore defines all the possible isomeric forms that the compounds of Formula (I) may possess. Unless otherwise mentioned or indicated, the chemical designation of compounds denotes the mixture of all possible stereochemically isomeric forms, said mixtures containing all diastereomers and enantiomers of the basic molecular structure. More in particular, stereogenic centers may have the R— or S-configuration; substituents on bivalent cyclic (partially) saturated radicals may have either the cis- or trans-configuration. Compounds encompassing double bonds can have an E or Z-stereochemistry at said double bond. Stereochemically isomeric forms of the compounds of Formula (I) are obviously intended to be embraced within the scope of this invention.

Following CAS nomenclature conventions, when two stereogenic centers of known absolute configuration are present in a molecule, an R or S descriptor is assigned (based on Cahn-Ingold-Prelog sequence rule) to the lowest-numbered chiral center, the reference center. R* and S* each indicate optically pure stereogenic centers with undetermined absolute configuration. If “α” and “ε” are used: the position of the highest priority substituent on the asymmetric carbon atom in the ring system having the lowest ring number, is arbitrarily always in the “α” position of the mean plane determined by the ring system. The position of the highest priority substituent on the other asymmetric carbon atom in the ring system (hydrogen atom in compounds according to Formula (I)) relative to the position of the highest priority substituent on the reference atom is denominated “α”, if it is on the same side of the mean plane determined by the ring system, or “β”, if it is on the other side of the mean plane determined by the ring system.

In the framework of this application, a compound according to the invention is inherently intended to comprise all isotopic combinations of its chemical elements. In the framework of this application, a chemical element, in particular when mentioned in relation to a compound according to Formula (I), comprises all isotopes and isotopic mixtures of this element, either naturally occurring or synthetically produced, either with natural abundance or in an isotopically enriched form. In particular, when hydrogen is mentioned, it is understood to refer to ¹H, ²H, ³H and mixtures thereof; when carbon is mentioned, it is understood to refer to ¹¹C, ¹²C, ¹³C, ¹⁴C and mixtures thereof; when nitrogen is mentioned, it is understood to refer to ¹³N, ¹⁴N, ¹⁵N and mixtures thereof; when oxygen is mentioned, it is understood to refer to ¹⁴O, ¹⁵O, ¹⁶O, ¹⁷O, ¹⁸O and mixtures thereof; and when fluor is mentioned, it is understood to refer to ¹⁸F, ¹⁹F and mixtures thereof.

A compound according to the invention therefore inherently comprises a compound with one or more isotopes of one or more element, and mixtures thereof, including a radioactive compound, also called radiolabelled compound, wherein one or more non-radioactive atoms has been replaced by one of its radioactive isotopes. By the term “radiolabelled compound” is meant any compound according to Formula (I), a pharmaceutically acceptable acid or base addition salt thereof, an N-oxide form thereof, or a quaternary ammonium salt thereof, which contains at least one radioactive atom. For example, a compound can be labelled with positron or with gamma emitting radio-active isotopes. For radioligand-binding techniques (membrane receptor assay), the ³H-atom or the ¹²⁵I-atom is the atom of choice to be replaced. For imaging, the most commonly used positron emitting (PET) radioactive isotopes are ¹¹C, ¹⁸F, ¹⁵O and ¹³N, all of which are accelerator produced and have half-lives of 20, 100, 2 and 10 minutes respectively. Since the half-lives of these radioactive isotopes are so short, it is only feasible to use them at institutions which have an accelerator on site for their production, thus limiting their use. The most widely used of these are ¹⁸F, ^{99m}Tc, ²⁰¹T1 and ¹²³I. The handling of these radioactive isotopes, their production, isolation and incorporation in a molecule are known to the skilled person.

In particular, the radioactive atom is selected from the group of hydrogen, carbon, nitrogen, sulfur, oxygen and halogen. Preferably, the radioactive atom is selected from the group of hydrogen, carbon and halogen.

In particular, the radioactive isotope is selected from the group of ³H, ¹¹C, ¹⁸F, ¹²²I, ¹²³I, ¹²⁵I, ¹³¹I, ⁷⁵Br, ⁷⁶Br, ⁷⁷Br and ⁸²Br. Preferably, the radioactive isotope is selected from the group of ³H, ¹¹C and ¹⁸F.

The numbering of the tetracyclic ring-system present in the compounds of Formula (I), as defined by Chemical Abstracts nomenclature is shown in the Formula below.

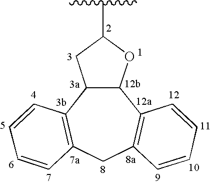

The compounds of Formula (I) have at least three stereogenic centers in their chemical structure, namely carbon atom 2, 3a and 12b. Said asymmetric center and any other asymmetric center which may be present, are indicated by the descriptors R and S.

The compounds of Formula (I) as prepared in the processes described below may be synthesized in the form of racemic mixtures of enantiomers that can be separated from one another following art-known resolution procedures. The racemic compounds of Formula (I) may be converted into the corresponding diastereomeric salt forms by reaction with a suitable chiral acid. Said diastereomeric salt forms are subsequently separated, for example, by selective or fractional crystallization and the enantiomers are liberated therefrom by alkali. An alternative manner of separating the enantiomeric forms of the compounds of Formula (I) involves liquid chromatography using a chiral stationary phase. Said pure stereochemically isomeric forms may also be derived from the corresponding pure stereochemically isomeric forms of the appropriate starting materials, provided that the reaction occurs stereospecifically. Preferably if a specific stereoisomer is desired, said compound would be synthesized by stereospecific methods of preparation. These methods will advantageously employ enantiomerically pure starting materials.

### Pharmacology

The compounds of the present invention show affinity for 5-HT₂ receptors, particularly for 5-HT_{2A} and 5-HT_{2C} receptors (nomenclature as described by D. Hoyer in “Serotonin (5-HT) in neurologic and psychiatric disorders” edited by M. D. Ferrari and published in 1994 by the Boerhaave Commission of the University of Leiden) and affinity for the D₂ receptor. The serotonin antagonistic properties of the present compounds may be demonstrated by their inhibitory effect in the “5-hydroxytryptophan Test on Rats” which is described in Drug Dev. Res., 13, 237-244 (1988).

The compounds of the present invention also have favorable physicochemical properties. For instance, they are chemically stable compounds.

In view of their capability to block 5-HT₂ receptors, and in particular to block 5-HT_{2A} and ⁵-HT_{2C} receptors, as well as the D₂ receptor the compounds according to the invention are useful as a medicine, in particular in the prophylactic and therapeutic treatment of conditions mediated through either of these receptors.

The invention therefore relates to a compound according to the general Formula (I), a pharmaceutically acceptable acid or base addition salt thereof, a stereochemically isomeric form thereof, an N-oxide form thereof, and a quaternary ammonium salt thereof, for use as a medicine.

The invention also relates to the use of a compound according to the general Formula (I), a pharmaceutically acceptable acid or base addition salt thereof, a stereo-chemically isomeric form thereof, an N-oxide form thereof, and a quaternary ammonium salt thereof, for the manufacture of a medicament for treating, either prophylactic or therapeutic or both, conditions mediated through the 5-HT₂, and/or D₂ receptors.

In view of these pharmacological and physicochemical properties, the compounds of Formula (I) are useful as therapeutic agents in the treatment or the prevention of central nervous system disorders like anxiety, bipolar disorders, sleep- and sexual disorders, psychosis, borderline psychosis, schizophrenia, migraine, personality disorders or obsessive-compulsive disorders, social phobias or panic attacks, organic mental disorders, mental disorders in children such as ADHD, aggression, memory disorders and attitude disorders in older people, addiction, obesity, bulimia and similar disorders. In particular, the present compounds may be used as anxiolytics, antipsychotics, anti-schizophrenia agents, anti-migraine agents and as agents having the potential to over-rule the addictive properties of drugs of abuse.

The compounds of Formula (I) may also be used as therapeutic agents in the treatment of motor disorders. It may be advantageous to use the present compounds in combination with classical therapeutic agents for such disorders.

The compounds of Formula (I) may also serve in the treatment or the prevention of damage to the nervous system caused by trauma, stroke, neurodegenerative illnesses and the like; cardiovascular disorders like high blood pressure, thrombosis, stroke, and the like; and gastrointestinal disorders like dysfunction of the motility of the gastrointestinal system and the like.

In view of the above uses of the compounds of Formula (I), it follows that the present invention also provides a method of treating warm-blooded animals suffering from such diseases, said method comprising the systemic administration of a therapeutic amount of a compound of Formula (I) effective in treating the above described disorders, in particular, in treating anxiety, psychosis, migraine and addictive properties of drugs of abuse.

The present invention thus also relates to compounds of Formula (I) as defined hereinabove for use as a medicine, in particular, the compounds of Formula (I) may be used for the manufacture of a medicament for treating anxiety, psychosis, migraine and addictive properties of drugs of abuse.

Those of skill in the treatment of such diseases could determine the effective therapeutic daily amount from the test results presented hereinafter. An effective therapeutic daily amount would be from about 0.01 mg/kg to about 10 mg/kg body weight, more preferably from about 0.05 mg/kg to about 1 mg/kg body weight.

The invention also relates to a pharmaceutical composition comprising a pharmaceutically acceptable carrier and, as active ingredient, a therapeutically effective amount of a compound according to the invention, in particular a compound according to Formula (I), a pharmaceutically acceptable acid or base addition salt thereof, a stereochemically isomeric form thereof, an N-oxide form thereof, and a quaternary ammonium salt thereof.

For ease of administration, the subject compounds may be formulated into various pharmaceutical forms for administration purposes. The compounds according to the invention, in particular the compounds according to Formula (I), a pharmaceutically acceptable acid or base addition salt thereof, a stereochemically isomeric form thereof, an N-oxide form thereof, and a quaternary ammonium salt thereof, or any sub-group or combination thereof may be formulated into various pharmaceutical forms for administration purposes. As appropriate compositions there may be cited all compositions usually employed for systemically administering drugs. To prepare the pharmaceutical compositions of this invention, an effective amount of the particular compound, optionally in addition salt form, as the active ingredient is combined in intimate admixture with a pharmaceutically acceptable carrier, which carrier may take a wide variety of forms depending on the form of preparation desired for administration. These pharmaceutical compositions are desirable in unitary dosage form suitable, in particular, for administration orally, rectally, percutaneously, by parenteral injection or by inhalation. For example, in preparing the compositions in oral dosage form, any of the usual pharmaceutical media may be employed such as, for example, water, glycols, oils, alcohols and the like in the case of oral liquid preparations such as suspensions, syrups, elixirs, emulsions and solutions; or solid carriers such as starches, sugars, kaolin, diluents, lubricants, binders, disintegrating agents and the like in the case of powders, pills, capsules and tablets. Because of their ease in administration, tablets and capsules represent the most advantageous oral dosage unit forms in which case solid pharmaceutical carriers are obviously employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. Injectable solutions, for example, may be prepared in which the carrier comprises saline solution, glucose solution or a mixture of saline and glucose solution. Injectable solutions containing compounds of Formula (I) may be formulated in an oil for prolonged action. Appropriate oils for this purpose are, for example, peanut oil, sesame oil, cottonseed oil, corn oil, soybean oil, synthetic glycerol esters of long chain fatty acids and mixtures of these and other oils. Injectable suspensions may also be prepared in which case appropriate liquid carriers, suspending agents and the like may be employed. Also included are solid form preparations that are intended to be converted, shortly before use, to liquid form preparations. In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. Said additives may facilitate the administration to the skin and/or may be helpful for preparing the desired compositions. These compositions may be administered in various ways, e.g., as a transdermal patch, as a spot-on, as an ointment. Acid or base addition salts of compounds of Formula (I) due to their increased water solubility over the corresponding base or acid form, are more suitable in the preparation of aqueous compositions.

It is especially advantageous to formulate the aforementioned pharmaceutical compositions in unit dosage form for ease of administration and uniformity of dosage. Unit dosage form as used herein refers to physically discrete units suitable as unitary dosages, each unit containing a predetermined quantity of active ingredient calculated to produce the desired therapeutic effect in association with the required pharmaceutical carrier. Examples of such unit dosage forms are tablets (including scored or coated tablets), capsules, pills, powder packets, wafers, suppositories, injectable solutions or suspensions and the like, and segregated multiples thereof.

Since the compounds according to the invention are potent orally administrable compounds, pharmaceutical compositions comprising said compounds for administration orally are especially advantageous.

In order to enhance the solubility and/or the stability of the compounds of Formula (I) in pharmaceutical compositions, it can be advantageous to employ α-, β- or γ-cyclodextrins or their derivatives, in particular hydroxyalkyl substituted cyclodextrins, e.g. 2-hydroxypropyl-β-cyclodextrin. Also co-solvents such as alcohols may improve the solubility and/or the stability of the compounds according to the invention in pharmaceutical compositions.

### Preparation

The compounds of formula (I) can generally be prepared by N-alkylating an intermediate compound of formula (II) with an intermediate compound of formula (III) wherein W is a suitable leaving group such as halo for example bromo, or an organo-sulfonyl group such as p-toluenesulfonyl.

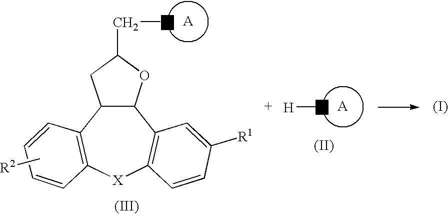

In the intermediate compounds (II) and (III), R¹, R², X and the cyclic moiety A are as defined in the compounds of formula (I). Said N-alkylation can conveniently be carried out in a reaction-inert solvent such as, for example, methanol, ethanol, tetrahydrofuran, methylisobutyl ketone, N,N-dimethylformamide, dimethylsulfoxide or acetonitrile and optionally in the presence of a suitable base such as calcium oxide. Stirring and elevated temperatures, for instance reflux temperature, may enhance the rate of the reaction.

Alternatively, said N-alkylation may also be performed using the procedure described by Monkovic et al. (J. Med. Chem. (1973), 16(4), p. 403-407) which involves the use of a pressurized reaction vessel.

The compounds of formula (I) may also be converted into each other following art-known transformation reactions, for example:
(a) a compound of formula (I) wherein R⁵ is C₁₋₆alkyl substituted with hydroxy may be converted into a corresponding compound of formula (I) in which R⁵ is C₁₋₆alkyl substituted with C₁₋₆alkyloxy by treatment with an organosulfonyl halide for example methanesulfonyl chloride, for example in the presence of a base such triethylamine, generally in a solvent such dichloromethane, to form the corresponding intermediate compound in which R⁵ is C₁₋₆alkyl substituted with organosulfonyloxy which is then treated with a methyl C₁₋₆alkanoate, generally in a suitable solvent such as ethanol;
(b) a compound of formula (I) wherein R⁵ is C₁₋₆alkyl substituted with hydroxy may be converted into a corresponding compound of formula (I) in which R⁵ is C₁₋₆alkyl substituted with C₁₋₆alkyl-carbonyloxy by acylation with a suitable acylating agent for example an acyl halide such as an acyl chloride, for example in the presence of a base such as triethylamine, generally in a solvent such as dichloromethane;
(c) a compound of formula (I) wherein R⁵ is hydrogen may be converted into a corresponding compound of formula (I) in which R⁵ is C₁₋₆alkylcarbonyl by acylation with a suitable acylating agent for example an acyl halide such as an acyl chloride, for example in the presence of a base such as triethylamine, generally in a solvent such as dichloromethane;
(d) a compound of formula (I) wherein R⁵ is hydrogen may be converted into a corresponding compound of formula (I) in which R⁵ is C₂alkyl substituted with both hydroxy and aryl in the 2-position, by treatment with an appropriate aryl-epoxide in a suitable solvent for example propanol;
(e) a compound of formula (I) wherein R⁵ is C₁₋₆alkyl substituted with hydroxy may be converted into a corresponding compound of formula (I) in which R⁵ is C₁₋₆alkyl substituted with C₁₋₆alkyloxy by treatment with an organosulfonyl halide for example methanesulfonyl chloride, for example in the presence of a base such as triethylamine, generally in a solvent such dichloromethane, to from the corresponding intermediate compound in which R⁵ is C₁₋₆alkyl substituted with organosulfonyloxy, which is then treated with an alkyloxy metal compound for example the sodium compound, generally in a suitable solvent such as methanol;
(f) a compound of formula (I) wherein R⁵ is hydrogen may be converted into a corresponding compound of formula (I) in which R⁵ is C₁₋₆alkyl optionally substituted with aryl, by treatment with a C₁₋₆alkyl (optionally substituted with aryl) aldehyde in the presence of polymer supported sodium cyanoborohydride (PS—CNBH₄Na) and polymer supported sulphonic acid (PS—SO₃H) in THF/acetic acid and CH₂Cl₂ and TFA;
(g) a compound of formula (I) wherein R⁵ is hydrogen may be converted into a corresponding compound of formula (I) in which R⁵ is C₁₋₆alkyl substituted with aryl and hydroxyl by treatment with an arylcarbonylalkyl halide such as a 2-arylcarbonylethyl halide, e.g. chloride, to form the corresponding intermediate compound in which R⁵ is C₁₋₆alkyl substituted with arylcarbonyl which is then reduced for example with sodium borohydride, generally in a solvent such as ethanol, to form the desired compound of formula (I); or
(h) a compound of formula (I) wherein R² is halo (for example iodo) may be converted into a corresponding compound of formula (I) in which R² is cyano by treatment with a cyanide compound, for example zinc cyanide, in the presence of a palladium compound such as Pd(PPh₃)₄, in a suitable solvent, for example N,N-dimethylformamide.

The intermediate compounds mentioned hereinabove are either commercially available or may be made following art-known procedures. For instance, intermediate compounds of formula (III) may be prepared according to the procedure described by Monkovic et al. (J. Med. Chem. (1973), 16(4), p. 403-407).

Alternatively, intermediate compounds of formula (III), said intermediate compounds being represented by formula (III-a), can also be prepared by reacting an epoxide derivative of formula (IV) with a Grignard reagent of formula (V) wherein X suitably is halo, thus forming an intermediate compound of formula (VI) which may subsequently be cyclized according to art-known methods such as the one described in Monkovic et al.

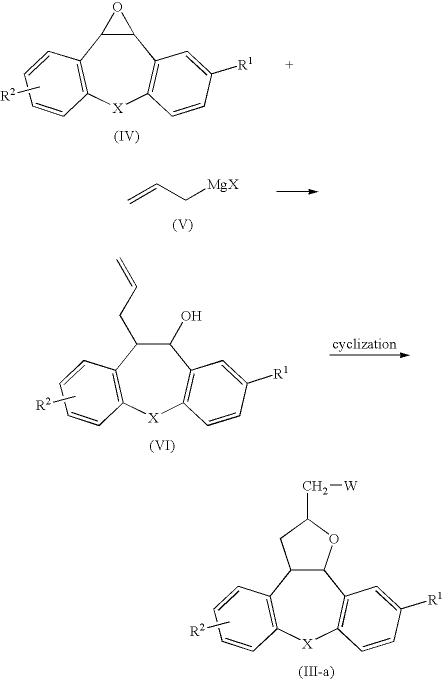

Epoxides of formula (IV) can be prepared using art-known procedures such as epoxidating an intermediate compound of formula (VII) with a suitable peroxide such as m-chloroperbenzoic acid.

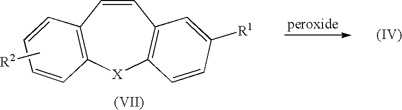

Pure stereochemically isomeric forms of the compounds of Formula (I) may be obtained by the application of art-known procedures. Diastereomers may be separated by physical methods such as selective crystallization and chromatographic techniques, e.g. counter-current distribution, liquid chromatography and the like.

### Experimental Part

Hereinafter, “DMF” is defined as N,N-dimethylformamide, “DCM” is defined as dichloromethane, “Et₃N” is defined as triethylamine, “EtOAc” is defined as ethyl acetate, “EtOH” is defined as ethanol, “MeOH” is defined as methanol and “THF” is defined as tetrahydrofuran.

### A. Preparation of the Intermediate Compounds

### EXAMPLE A1

### a) Preparation of Intermediate Compound 1

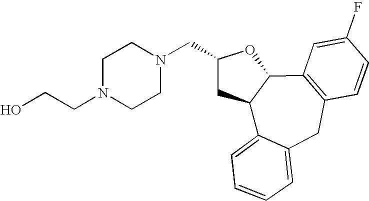 [0089] [2R-(2α, 3aα, 12bβ)]

11 -Fluoro-3,3a,8,12b-tetrahydro-N-methyl-2H-dibenzo[3,4:6,7]cyclohepta[1,2-b] furan-2-methanamine [2R-(2α, 3aα, 12bβ)] (described in WO 03/048146) (0.0114 mol) and 1-piperazine-ethanol (0.0342 mol) were irradiated under microwave conditions (power: 500 Watt; 150° C; 15 min). Then, the resulting mixture was diluted with EtOAc. The organic solution was washed with water, dried, filtered and the solvent evaporated under reduced pressure. The residue was purified by short open column chromatography over silica gel (eluent: DCM/MeOH 97/3). The product fractions were collected and the solvent was evaporated, yielding 2.5 g of intermediate compound 1 as an orange oil which was used in next reaction step without further purification.

### b) Preparation of Intermediate Compound 2

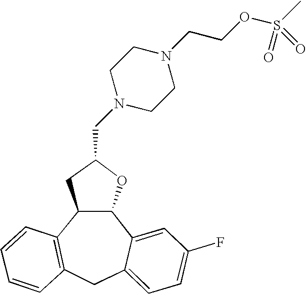 [0092] [2R-(2α, 3aα, 12bβ)]

Methanesulfonyl chloride (0.00225 mol) was added to a solution of intermediate compound 1 (0.0015 mol) in Et₃N (0.42 ml) and dry DCM (10 ml), stirred at 0° C. The reaction mixture was stirred for 16 hours at room temperature. Water was added and the mixture was stirred. The organic layer was separated, dried, filtered and the solvent evaporated. The residue was purified by short open column chromatography over silica gel (eluent: DCM/MeOH 98/8). The product fractions were collected and the solvent was evaporated, yielding 0.390 g of intermediate compound 2 .

### EXAMPLE A2

### Preparation of Intermediate Compound 3

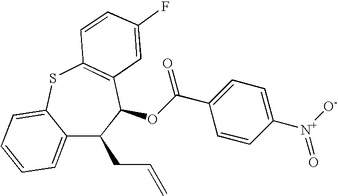

trans isomer, racemic mixture

A solution of triphenylphosphine (0.02032 mol) in THF (100 ml) was stirred at 0° C. under N₂, then bis(1-methylethyl)diazenedicarboxylate (0.01992 mol) was added and the resulting suspension was stirred for 30 min. A solution of cis-8-fluoro-10,11-dihydro-11-(2-propenyl)-dibenzo[b,f]thiepin-10-ol (described in J. Med. Chem. 2005, 48, 1709) (0.01016 mol) and 4-nitro-benzoic acid (0.02032 mol) in THF was added dropwise and then the reaction mixture was gradually warmed to room temperature and stirred for 16 hours. The solvent was evaporated and the residue was purified by column chromatography (eluent: EtOAc/heptane 1/9). The product fractions were collected and the solvent was evaporated, yielding 3.96 g (89 %) of intermediate compound 3 .

### Preparation of Intermediate Compound 4

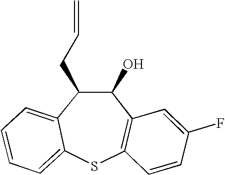

trans isomer, racemic mixture

A mixture of intermediate compound 3 (0.0121 mol) and lithium hydroxide (0.0127 mol) in THF and water was stirred for 16 hours at room temperature and then the solvent was evaporated. The obtained residue was dissolved in DCM and washed with water and with brine, then the organic layer was dried and the solvent was evaporated, yielding 3.96 g (colorless oil) of intermediate compound 4, used in the next reaction step without further purification.

### Preparation of Intermediate Compound 5

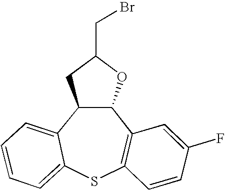 [0099] 2RS-(2β, 3aα, 12bβ)+2RS-(2α, 3aα, 12bβ)]

Intermediate compound 4 (0.00387 mol) was dissolved in chloroform (120 ml) at room temperature and the mixture was stirred at 0° C. for 3 minutes, then pyridinium tribromide (0.0039 mol) was added portionwise. After 10 minutes at 0° C., the cold bath was removed and the reaction mixture was stirred for 1 hour. Water was added and the layers were separated. The organic layer was dried (Na₂SO₄) and the solvent was evaporated (vac.) (¹H-NMR: mixture of diastercoisomers 73/27 at position C2). The residue was purified by radial chromatography (eluent: heptane/DCM mixtures). The product fractions were collected and the solvent was evaporated, yielding 1.00 g (colorless oil, trans fused isomer) of intermediate compound 5 .

### EXAMPLE A3

### Preparation of Intermediate Compound 6

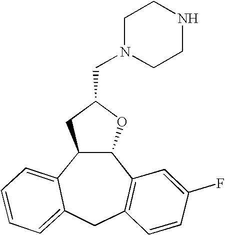

[2R-(2α, 3aα, 12bβ)]

A mixture of 11 -fluoro-3,3a,8,12b-tetrahydro-2H-dibenzo[3,4:6,7]cyclohepta[1,2-b] furan-2-methanol 4-methylbenzenesulfonate [2R-(2α, 3aα, 12bβ)] as described in WO 03/048146, (0.023 mol), piperazine (0.23 mol) and calcium oxide (2.3 mol) in THF was stirred and heated for 16 hours at 140° C. (oil bath temperature) in a Parr reactor vessel, then the reaction mixture was cooled to room temperature. The solids were filtered off and the filtrate was evaporated. The residue was taken up in EtOAc and was washed two times with water. The organic layer was dried (Na₂SO₄), filtered and the solvent was evaporated (vac.), yielding intermediate compound 6 as a brown oil.

### EXAMPLE A4

### Preparation of Intermediate Compound 7

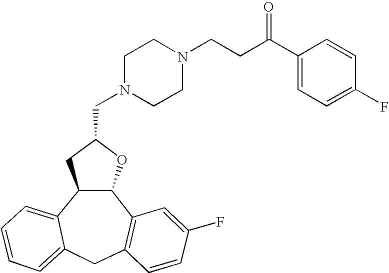 [0104] .C₂H₂O₄ (1:1) [2R-(2α, 3aα, 12bβ)]

A mixture of intermediate compound 6 (0.00284 mol), 3-chloro-1-(4-fluorophenyl)-1-propanone (0.00568 mol) and potassium carbonate (0.0057 mol) in acetonitrile (q.s.) was irradiated under microwave conditions at 250° C. for 20 minutes. The solution was concentrated under reduced pressure and the residue was dissolved in DCM, washed with water and with brine, then dried. The solvent was evaporated and the residue was purified by short open column chromatography over silica gel (eluent: DCM/MeOH 98/2). The product fractions were collected and the solvent was evaporated. A part of this residue (1.2 g, 84%) was converted into the ethanedioate salt (1:1) by treatment with oxalic acid in diethylether. The resulting precipitate was filtered off, washed with cold diethylether and dried, yielding 0.032 g of intermediate compound 7.

### EXAMPLE A5

### a) Preparation of Intermediate Compound 8

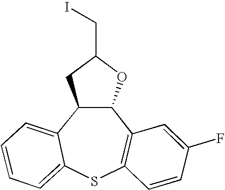 [0107] 1:1 mixture of diastereoisomers
2RS-(2β, 3aα, 12bβ)+2RS-(2α, 3aα, 12bβ)]

A mixture of intermediate compound 4 (0.033 mol) and iodine bis(pyridine)-tetrafluoroborate (0.033 mol) in DCM (200 ml) was stirred for one hour at room temperature. The reaction mixture was washed with a saturated aqueous Na₂S₂O₃ solution, with 2N HCl, with water and with brine, then the organic layer was dried, filtered and the solvent evaporated, yielding 4.375 g (32%) of intermediate compound 8, used in next reaction step without further purification.

### b) Preparation of Intermediate Compound 9

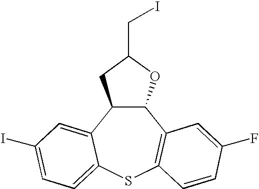 [0111] 2RS-(2β, 3aα, 12bβ)+2RS-(2α, 3aα, 12bβ)]

A mixture of intermediate compound 8 (0.0106 mol), iodine bis(pyridine)-tetrafluoroborate (0.0117 mol) and triflic acid (0.0212 mol) in DCM (50 ml) was stirred for one hour at room temperature, under N₂ atmosphere. The reaction mixture was washed with Na₂S₂O₃ (2×50 ml), and with brine (2×50 ml). The organic layer 5 was separated, dried (Na₂SO₄), filtered and the solvent was evaporated. The residue (oil) was purified by short open column chromatography over silica gel (eluent: heptane/EtOAc 9/1). The product fractions were collected and the solvent was evaporated, yielding 3.9 g (68%) of intermediate compound 9.

### c) Preparation of Intermediate Compound 10

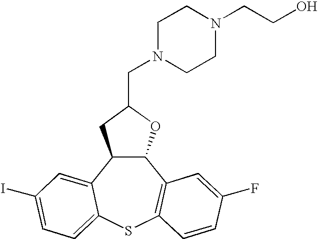 [0114] 2RS-(2β, 3aα, 12bβ)+2RS-(2α, 3aα, 12bβ)]

A mixture of intermediate compound 9 (0.0024 mol), 1-piperazine-ethanol (0.0024 mol) and calcium oxide (2 g) in THF (20 ml) was stirred for 16 hours at 120° C. in a Parr pressure vessel. After cooling to room temperature, the resultant suspension was filtered through Celite. The filtrate was evaporated under reduced pressure. The residue was redissolved in DCM. The organic solution was washed with an aqueous NaHCO₃-solution, with water, with brine, then separated, dried (Na2SO₄), filtered and the solvent was evaporated. The residue (oil) was purified by short open column chromatography over silica gel. The product fractions were collected and the solvent was evaporated, yielding 0.876 g of intermediate compound 10, which was used in next reaction step, without further purification.

### B. Preparation of the Final Compounds

### EXAMPLE B1

### Preparation of Final Compound 12

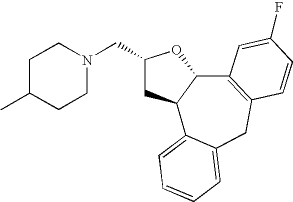 [0117] .C₂H₂O₄ (1:1) [2R-(2α, 3aα, 12bβ)]

A mixture of (2R,3aR,12bS)-11-fluoro-3,3a,8,12b-tetrahydro-2H-dibenzo[3,4:6,7]cyclohepta[1,2-b]furan-2-methanol-4-methylbenzenesulfonate,(0.00045 mol), described in WO 03/048146, 4-methyl-piperidine (0.00228 mol) and calcium oxide (0.00228 mol) in acetonitrile (q.s.) was heated in a sealed tube at 100° C. for 3 days, then the suspension was filtered and the filtrate was evaporated under reduced pressure. The residue was purified by short open column chromatography (eluent: DCM/MeOH 98.5/1.5). The product fractions were collected and the solvent was evaporated. The residue was converted into the ethanedioate salt and then the resulting salt was collected, yielding 0.164 g (98%) of final compound 12.

### EXAMPLE B2

### Preparation of Final Compound 34

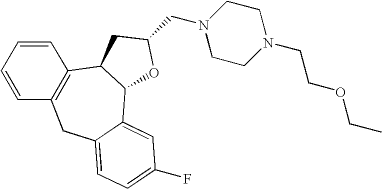 [0120] .C₂H₂O₄ (1:2) [2R-(2α, 3aα, 12bβ)]

A mixture of intermediate compound 2 (0.00019 mol) and sodium ethoxyde (0.0019 mol) in EtOH (10 ml) was stirred in a microwave oven at 100° C. for 20 minutes. Water was added. The mixture was concentrated. DCM was added and the mixture was shaken. The separated organic layer was dried, filtered and the solvent was evaporated. The residue was purified by short open column chromatography over silica gel (eluent: DCM/MeOH 98/2). The desired fractions were collected and the solvent was evaporated. The residue was converted into the oxalate salt, yielding 0.128 g of final compound 34.

### EXAMPLE B3

### Preparation of Final Compound 5

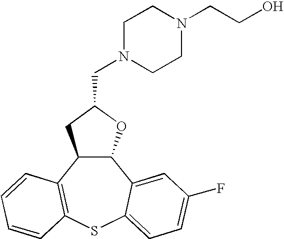 [0123] .C₂H₂O₄ (1:2) [2RS-(2α, 3aα, 12b β)]

A mixture of intermediate compound 5 (0.003 mol), 1-piperazine-ethanol (0.03 mol) and calcium oxide (0.03 mol) in THF (20 ml) was heated at 140° C. for 10 hours in a pressure vessel, then filtered over decalite. The filtrate solvent was evaporated under reduced pressure. The residue was dissolved in EtOAc, washed with water and brine and dried. This fraction was purified by high performance liquid chromatography over silica gel (eluent: DCM/(MeOH/NH₃) 99/1). The desired fractions were collected and the solvent was evaporated. The residue (0.045 g) was converted into the oxalate salt, yielding 0.047 g of final compound 39.

### EXAMPLE B4

### Preparation of Final Compound 5

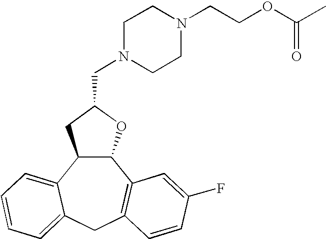 [0126] .C₂H₂O₄ (1:1) [2R-(2α, 3aα, 12bβ)]

A mixture of 1-piperazine ethanol, 4-[[(2R,3aR,12bS)-11-fluoro-3,3a,8,12b-tetrahydro-2H-dibenzo[3,4:6,7]cyclohepta[1,2-b]furan-2-yl]methyl] (0.000504 mol), described in WO 99/19317, acetyl chloride (0.000605 mol) and Et₃N (0.00101 mol) in DCM (10 ml) was stirred at room temperature for 16 hours, then the reaction mixture was washed with water, dried and the solvent was evaporated. The residue was purified by short open column chromatography over silica gel (eluent: DCM/MeOH 98/2). The product fractions were collected and the solvent was evaporated. The residue was converted into the ethanedioic acid salt (1:1). The precipitate was filtered off, then dried, yielding 0.187 g of final compound 5 .

### EXAMPLE B5

### Preparation of Final Compound 22

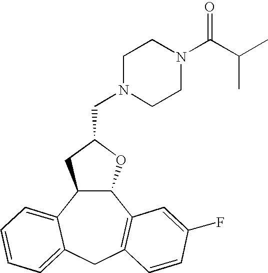 [0129] .C₂H₂O₄ (1:1) [2R-(2α, 3α, 12bβ)]

2-Methyl-propanoyl chloride (1.1 equiv) and Et₃N (2 equiv) were added to a solution of intermediate compound 6 (0.000567 mol, 1 equiv) in DCM (3 ml), stirred at room temperature. The reaction mixture was stirred for 6 hours at room temperature. Poly-styrene-trisamine (1 equiv) was added to scavenge excess of 2-methyl-propanoyl chloride, while stirring for one hour. Then, the resin was filtered off and the filtrate was evaporated in vacuo. The residue was purified by short open column chromatography over silica gel. The product fractions were collected and the solvent was evaporated. The free base residue was dissolved in diethyl ether and converted into the ethanedioic acid salt (1:1). The precipitate was filtered off and dried, yielding 0.04742 g of final compound 22.

### EXAMPLE B6

### Preparation of Final Compound 27

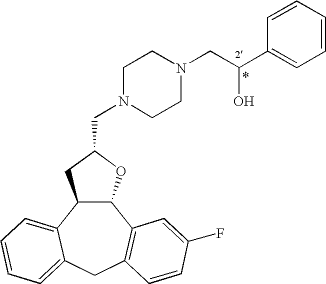 [0132] .C₂H₂O₄ (1:2) [2R-(2α, 3aα, 12bβ)] (2′RS)

A mixture of intermediate compound 6 (0.000567 mol ) and phenyl-oxirane (2 equiv) in 2-propanol (20 ml) was stirred overnight at 130° C. (oil-bath temperature). The reaction mixture was cooled to room temperature. The solvent was evaporated. The residue was purified by HPLC. The product fractions were collected and the solvent was evaporated, yielding 0.05628 g of the free base compound. The free base residue was dissolved in diethyl ether and converted into the ethanedioic acid salt (1:1). The precipitate was filtered off and dried, yielding final compound 27.

### EXAMPLE B7

### Preparation of Final Compound 35

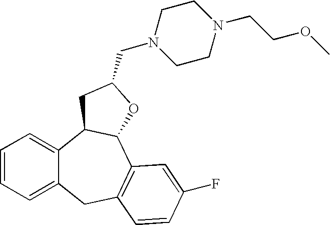 [0135] .C₂H₂O₄ (1:2) [2R-(2α, 3aα, 12bβ)]

A mixture of intermediate compound 2 (0.00021 mol), NaOMe/MeOH (3 ml) and MeOH (10 ml) was mixed and heated for 15 minutes at 90° C. under microwave irradiation (500 W) and the reaction mixture was quenched with water, then extracted two times with DCM. The organic extracts were combined, dried and the solvent was evaporated (vac.). The residue was purified by short open column chromatography. The product fractions were collected and the solvent was evaporated. The residue was treated with oxalic acid in diethyl ether and converted into the ethanedioate salt (1:2). The resulting precipitate was collected and dried, yielding final compound 35.

### EXAMPLE B8

### Preparation of Final Compound 40

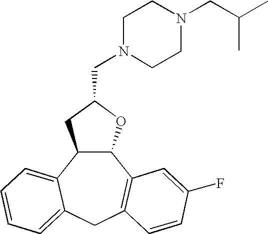 [0138] .C₂HF₃O₂ (1:1) [2R-(2α, 3aα, 12bβ)]

A mixture of intermediate compound 6 (0.085 g, 0.2414 mmol) and 2-methyl-propanal (1.5 eq, 0.3621 mmol) in a mixture of THF/acetic acid (4 mL/0.2 mL). To this solution polymer supported sodium borohydride (2.5 eq) was added. The reaction mixture was shaken for 20 hours at room temperature. The solids were filtered off and the volatiles were evaporated in vacuum. The residue thus obtained was taken up in MeOH (4 mL) and polymer supported SO₃H (1.5 eq) was added. The mixture was shaken at room temperature for 20 hours. The resin was filtered off and washed two times with MeOH and two times with DCM. MeOH saturated with NH₃ was added to the resin and shaken for 5 hours. The resin was filtered off and the solution was evaporated affording the corresponding product as the pure free base. The free base was treated with a solution of trifluoroacetic acid in DCM, yielding final compound 40.

### EXAMPLE B9

### Preparation of Final Compound 46

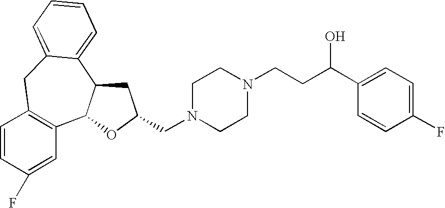 [0141] .C₂H₂O₄ [2R-(2α, 3aα, 12bβ)] [3′RS]

A mixture of intermediate compound 7 (0.00030 mol) and sodium tetrahydroboride (0.003 mol) in EtOH (5 ml) was stirred for 10 hours at room temperature and then the reaction mixture was partitioned between water/DCM. The aqueous layer was extracted several times with DCM; the organic layers were combined, extracted with brine, dried (Na₂SO₄) and filtered off. The solvent was evaporated under reduced pressure and the residue was converted into the ethanedioate salt (1:1). The resulting precipitate was filtered off, washed and dried, yielding 0.110 g of final compound 46.

### EXAMPLE B10

### Preparation of Final Compound 49

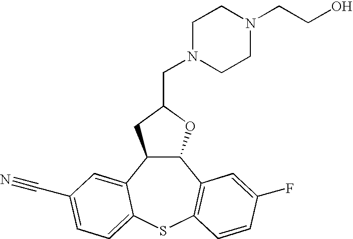 [0144] Mixture (15:85) of two trans fused diastereoisomers [85%2RS-(2β, 3aα, 12bβ)+(13%) 2RS-(2α, 3aα, 12bβ)]

A mixture of intermediate compound 10 (0.0016 mol), zinc cyanide (0.0010 mol) and tetrakis(triphenylphosphine)-palladium (0.00017 mol) in DMF (10 ml, previously deoxygenated) was stirred at room temperature and then heated at 120° C. (from 0° C. to 120° C. in 5 min.) for 15 minutes under microwave conditions. The mixture was filtered and the organic solvent (DMF) was evaporated. The residue was purified by radial chromatography, then the product fractions were collected and the solvent was evaporated, yielding final compound 49.

The final compounds prepared hereinunder all are mixtures of isomeric forms, unless otherwise specified.

Table 1 lists final compounds of Formula (I) which were prepared according to one of the above examples. Table 2 shows LCMS data for a selected set of final compounds.

TABLE 1

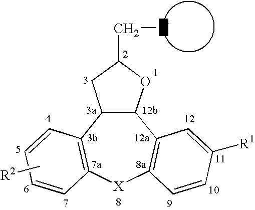

| Co.No. | Ex.No. | X | 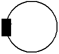 | R¹ | R² | Stereochemistry/Salt Form |
|---|---|---|---|---|---|---|
| 7 | B1 | —O— | 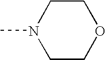 | —F | —H | •C₂H₂O₄, [2RS(2α, 3aα, 12bα)] |
| 8 | B1 | —O— | 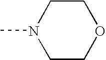 | —F | —H | •C₂H₂O₄, [2RS(2α, 3aβ, 12bβ)] |
| 6 | B1 | —O— | 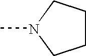 | —Cl | —H | •C₂H₂O₄, [2RS(2α, 3aα, 12bβ)] |
| 13 | B1 | —O— | 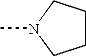 | —F | —H | •C₂H₂O₄, [2RS(2α, 3aα, 12bα)] |
| 19 | B1 | —O— | 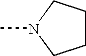 | —F | —H | •C₂H₂O₄, [2RS-(2α, 3aβ, 12bβ)] |
| 14 | B1 | —O— | 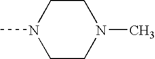 | —F | —H | •C₂H₂O₄, [2RS(2α, 3aα, 12bα)] |
| 18 | B1 | —O— | 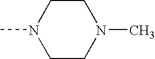 | —F | —H | •C₂H₂O₄, [2RS-(2α, 3aβ, 12bβ)] |
| 15 | B1 | —O— | 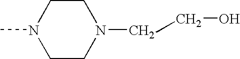 | —F | —H | [2RS(2α, 3aα, 12bα)] |
| 21 | B1 | —O— | 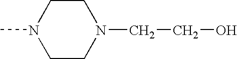 | —OCH₃ | —H | •C₂H₂O₄, [2RS-(2α, 3aα, 12bα)] |
| 25 | B1 | —O— | 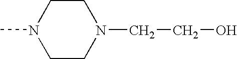 | —OCH₃ | —H | •C₂H₂O₄, [2RS-(2α, 3aβ, 12bβ)] |
| 26 | B1 | —O— | 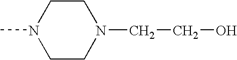 | —Br | —H | •C₂H₂O₄, [2RS-(2α, 3aα, 12bα)] |
| 3 | B1 | —S— | 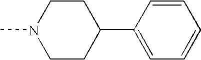 | —H | —H | Mixture (15:85) of two transfused diastereoisomers[(15%) 2RS-(2α, 3aβ, 12bα) +(85%) 2RS-(2α, 3aα, 12bβ)] |
| 2 | B1 | —S— | 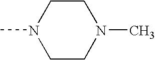 | —H | —H | Mixture (15:85) of two transfused diastereoisomers.2 C₂H₂O₄,[(15%) 2RS-(2α, 3aβ, 12bα) +(85%) 2RS-(2α, 3aα, 12bβ)] |
| 37 | B3 | —S— | 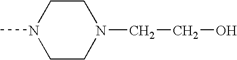 | —F | —H | [2RS-(2β, 3aα, 12bβ)] |
| 38 | B3 | —S— | 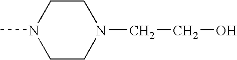 | —F | —H | .2 C₂H₂O₄, [2RS-(2β, 3aα, 12bβ)] |
| 39 | B3 | —S— | 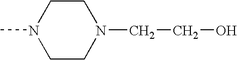 | —F | —H | .2 C₂H₂O₄, [2RS-(2α, 3aα, 12bβ)] |
| 49 | B10 | —S— | 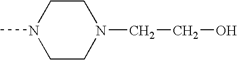 | —F | 5-CN | Mixture (15:85) of two transfused diastereoisomers[(60%) 2RS-(2β, 3aα, 12bβ) +(40%) 2RS-(2α, 3aα, 12bβ)] |
| 4 | B1 | —CH₂— | 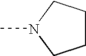 | —F | —H | •C₂H₂O₄, [2R-(2α, 3aα, 12bβ)] |
| 16 | B1 | —CH₂— | 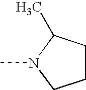 | —F | —H | •C₂H₂O₄, [2R-(2α, 3aα, 12bβ)],mixture of two diastereoisomers 1:1 |
| 17 | B1 | —CH₂— | 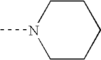 | —F | —H | •C₂H₂O₄, [2R-(2α, 3aα, 12bβ)] |
| 12 | B1 | —CH₂— | 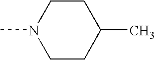 | —F | —H | •C₂H₂O₄, [2R-(2α, 3aα, 12bβ)] |
| 11 | B1 | —CH₂— | 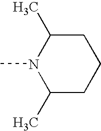 | —F | —H | •C₂H₂O₄, [2R-(2α, 3aα, 12bβ)] |
| 9 | B1 | —CH₂— | 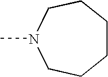 | —F | —H | [2R-(2α, 3aα, 12bβ)] |
| 10 | B1 | —CH₂— | 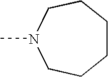 | —F | —H | •C₂H₂O₄, [2R-(2α, 3aα, 12bβ)] |
| 23 | B1 | —CH₂— | 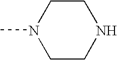 | —F | —H | [2R-(2α, 3aα, 12bβ)] |
| 24 | B1 | —CH₂— | 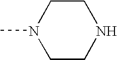 | —F | —H | .2 C₂H₂O₄, [2R-(2α, 3aα, 12bβ)] |
| 40 | B8 | —CH₂— | 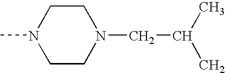 | —F | —H | •C₂HF₃O₂, [2R-(2α, 3aα, 12bβ)] |
| 29 | B6 | —CH₂— | 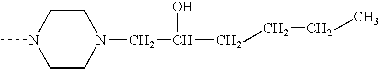 | —F | —H | .2 C₂H₂O₄, [2R-(2α, 3aα, 12bβ)](2′RS) |
| 30 | B6 | —CH₂— | 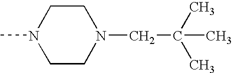 | —F | —H | .2 C₂H₂O₄, [2R-(2α, 3aα, 12bβ)] |
| 45 | B8 | —CH₂— | 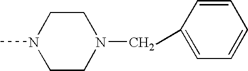 | —F | —H | •C₂HF₃O₂, [2R-(2α, 3aα, 12bβ)] |
| 42 | B8 | —CH₂— | 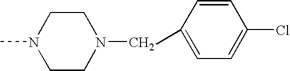 | —F | —H | •C₂HF₃O₂, [2R-(2α, 3aα, 12bβ)] |
| 47 | B8 | —CH₂— | 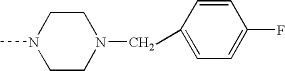 | —F | —H | •C₂HF₃O₂, [2R-(2α, 3aα, 12bβ)] |
| 44 | B8 | —CH₂— | 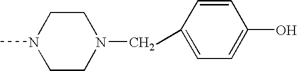 | —F | —H | •C₂HF₃O₂, [2R-(2α, 3aα, 12bβ)] |
| 43 | B8 | —CH₂— | 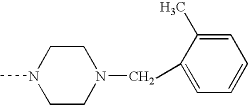 | —F | —H | •C₂HF₃O₂, [2R-(2α, 3aα, 12bβ)] |
| 41 | B8 | —CH₂— | 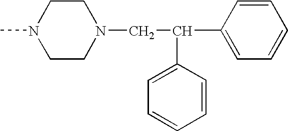 | —F | —H | •C₂HF₃O₂, [2R-(2α, 3aα, 12bβ)] |
| 48 | B8 | —CH₂— | 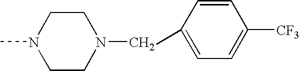 | —F | —H | •C₂HF₃O₂, [2R-(2α, 3aα, 12bβ)] |
| 27 | B6 | —CH₂— | 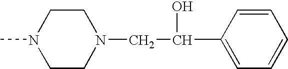 | —F | —H | .2 C₂H₂O₄, [2R-(2α, 3aα, 12bβ)][2′RS] |
| 46 | B6 | —CH₂— | 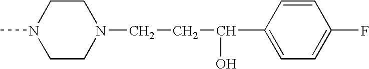 | —F | —H | •C₂H₂O₄, [2R-(2α, 3aα, 12bβ)][3′RS] |
| 28 | B6 | —CH₂— | 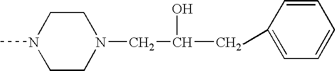 | —F | —H | .2 C₂H₂O₄, [2R-(2α, 3aα, 12bβ)][2′RS] |
| 22 | B5 | —CH₂— | 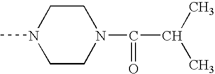 | —F | —H | •C₂H₂O₄, [2R-(2α, 3aα, 12bβ)] |
| 5 | B4 | —CH₂— | 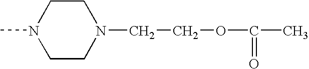 | —F | —H | •C₂H₂O₄, [2R-(2α, 3aα, 12bβ)] |
| 31 | B5 | —CH₂— | 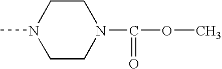 | —F | —H | •C₂H₂O₄, [2R-(2α, 3aα, 12bβ)] |
| 20 | B1 | —CH₂— | 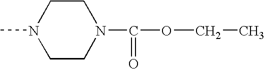 | —F | —H | •C₂H₂O₄, [2R-(2α, 3aα, 12bβ)] |
| 32 | B5 | —CH₂— | 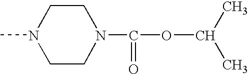 | —F | —H | •C₂H₂O₄, [2R-(2α, 3aα, 12bβ)] |
| 33 | B5 | —CH₂— | 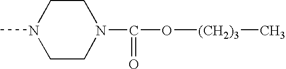 | —F | —H | •C₂H₂O₄, [2R-(2α, 3aα, 12bβ)] |
| 34 | B2 | —CH₂— | 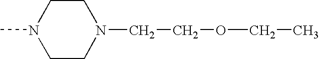 | —F | —H | .2 C₂H₂O₄, [2R-(2α, 3aα, 12bβ)] |
| 35 | B7 | —CH₂— | 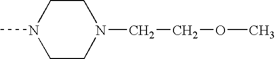 | —F | —H | .2 C₂H₂O₄, [2R-(2α, 3aα, 12bβ)] |
| 36 | B7 | —CH₂— | 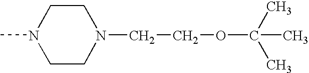 | —F | —H | .2 C₂H₂O₄, [2R-(2α, 3aα, 12bβ)] |

### Analytical Data

The LCMS data shown in Table 2 have been obtained by the following method:

The HPLC gradient was supplied by a HP 1100 from Agilent with a column heater set at 40° C. Flow from the column was passed through photodiode array (PDA) detector and then split to a Light Scattering detector (ELSD) and to a Waters-Micromass Time of Flight (ToF) mass spectrometer with an electrospray ionization source operated simultaneously in positive and negative ionization mode.

Reversed phase HPLC was carried out on a XDB-C18 cartridge (3.5 μm, 4.6×30 mm) from Agilent, with a flow rate of 1 ml/min. Three mobile phases (mobile phase A: 0.5 g/l ammoniumacetate solution, mobile phase B: acetonitrile; mobile phase C: methanol) were employed to run a gradient condition from 80% A, 10% B,10% C to 50% B and 50% C in 6.0 min., to 100% B at 6.5 min., kept till 7.0 min and reequilibrated with 80% A, 10% B and 10% C at 7.6 min. that was kept till 9.0 min. An injection volume of 5 μL was used.

High Resolution Mass spectra were acquired by scanning from 100 to 750 in 1 s using a dwell time of 1 s. The capillary needle voltage was 3 kV and the source temperature was maintained at 140° C. Nitrogen was used a the nebulizer gas. Cone voltage was 30 V for both positive and negative ionization mode. Leucine-enkephaline was the reference used for the lock spray. Data acquisition was performed with a Waters-Micromass MassLynx-Openlynx data system.

Data from the spectroscopic assays of the final compounds according to the invention are given below in Table 2; the symbol “-” in the relevant column indicates that no value was determined. The parent peak mass corresponds to the mass of the free base +H⁺.

TABLE 2

| | | Parent | Main Fragment/ |
|---|---|---|---|
| Co. | Retention | peak mass | Adduct |
| No. | time (min.) | (ES⁺) | (ES⁺) |
| 1 | 6.24 | 324 | 223 |
| 4 | 4.13 | 338 | 114 |
| 5 | 5.33 | 439 | — |
| 7 | 4.87 | 356 | 199 |
| 8 | 4.81 | 356 | 199 |
| 10 | 5.06 | 366 | 142 |
| 11 | 4.84 | 380 | 156 |
| 12 | 5.56 | — | — |
| 13 | 3.71 | 340 | — |
| 14 | 4.18 | 369 | — |
| 15 | 3.07 | 399 | — |
| 16 | 4.42/4.75 | 352 | 128 |
| 18 | 4.01 | 369 | 199 |
| 19 | 3.73 | 340 | 199 |
| 21 | 3.96 | 411 | 433 |
| 22 | 5.8 | 423 | 455 |
| 24 | 3.57 | 353 | — |
| 27 | 6.01 | 473 | 495 |
| 28 | 6.09 | 487 | 509 |
| 29 | 6.15 | 453 | 475 |
| 30 | 5.61 | 425 | 477 |
| 31 | 5.84 | 411 | — |
| 32 | 6.29 | 439 | — |
| 33 | 6.49 | 453 | — |
| 34 | 4.70 | 425 | 477 |
| 35 | 4.2 | 411 | 433 |
| 36 | 5.27 | 453 | 475 |
| 37 | 4.97/5.05 | 415 | 215 |
| 39 | 4.94/5.07 | 415 | 215 |
| 41 | 6.7 | 533 | 555 |
| 42 | 6.65 | 477 | — |
| 43 | 6.70 | 457 | — |
| 44 | 5.48 | 457 | — |
| 45 | 6.42 | 443 | — |
| 46 | 5.82 | 505 | 365 |
| 47 | 6.57 | 461 | — |
| 49 | 4.68 | 440 | — |

### C. Pharmacological Data

### EXAMPLE C.1

### In vitro Binding Affinity for 5-HT_{2A} and 5-HT_{2C} Receptors

The interaction of the compounds of Formula (I) with 5-HT_{2A} and 5-HT_{2C} receptors was assessed in in vitro radioligand binding experiments. In general, a low concentration of a radioligand with a high binding affinity for the receptor is incubated with a sample of a tissue preparation enriched in a particular receptor (1 to 5 mg tissue) in a buffered medium (0.2 to 5 ml). During the incubation, the radioligands bind to the receptor. When equilibrium of binding is reached, the receptor bound radioactivity is separated from the non-bound radioactivity, and the receptor bound activity is counted. The interaction of the test compounds with the receptors is assessed in competition binding experiments. Various concentrations of the test compound are added to the incubation mixture containing the tissue preparation and the radioligand. Binding of the radioligand will be inhibited by the test compound in proportion to its binding affinity and its concentration. The affinities of the compounds for the 5-HT₂ receptors were measured by means of radioligand binding studies conducted with: (a) human cloned 5-HT_{2A} receptor, expressed in L929 cells using [¹²⁵I]R91150 as radioligand and (b) human cloned 5-HT_{2C} receptor, expressed in CHO cells using [³H]mesulergine as radioligand.

### EXAMPLE C.2

### In vitro Binding Affinity for Human D2_{L} Receptor

Frozen membranes of human Dopamine D2_{L} receptor-transfected CHO cells were thawed, briefly homogenized using an Ultra-Turrax T25 homogenizer and diluted in Tris-HCl assay buffer containing NaCl, CaCl₂, MgCl₂, KCl (50, 120, 2, 1, and 5 mM respectively, adjusted to pH 7.7 with HCl) to an appropriate protein concentration optimized for specific and non-specific binding. Radioligand [³H]Spiperone (NEN, specific activity ˜70 Ci/mmol) was diluted in assay buffer at a concentration of 2 nmol/L. Prepared radioligand (50 μl), along with 50 μl of either the 10% DMSO control, Butaclamol (10⁻⁶ mol/l final concentration), or compound of interest, was then incubated (30 min, 37° C.) with 400 μl of the prepared membrane solution. Membrane-bound activity was filtered through a Packard Filtermate harvester onto GF/B Unifilter plates and washed with ice-cold Tris-HCl buffer (50 mM; pH 7.7; 6×0.5 ml). Filters were allowed to dry before adding scintillation fluid and counting in a Topcount scintillation counter. Percentage specific bound and competition binding curves were calculated using S-Plus software (Insightful).

The results are given in Table 3 below in terms of pIC₅₀ values for the respective compounds.

TABLE 3

| Co. No. | 5-HT_{2A} | 5-HT_{2C} | D2_{L} |
|---|---|---|---|
| 46 | 8.29 | 8.07 | 8.95 |
| 12 | 8.41 | 9.00 | 8.87 |
| 39 | 9.16 | 8.16 | 8.70 |
| 34 | 9.07 | 8.30 | 8.60 |
| 44 | 7.97 | 7.52 | 8.56 |
| 35 | 8.82 | 8.13 | 8.51 |
| 36 | 9.02 | 8.26 | 8.43 |
| 27 | 8.02 | 7.69 | 8.39 |
| 28 | 8.32 | 7.97 | 8.34 |
| 17 | 8.72 | 9.00 | 8.23 |
| 29 | 8.00 | 7.63 | 8.22 |
| 5 | 7.84 | 7.72 | 8.13 |
| 38 | 8.67 | 7.56 | 8.11 |
| 45 | 7.73 | 7.34 | 8.10 |
| 47 | 7.51 | 6.89 | 8.04 |
| 40 | 7.57 | 7.28 | 7.91 |
| 30 | 8.01 | 7.43 | 7.86 |
| 31 | 8.33 | 7.86 | 7.85 |
| 10 | 8.01 | 8.51 | 7.79 |
| 33 | 7.93 | 7.42 | 7.77 |
| 26 | 7.36 | 6.40 | 7.74 |
| 32 | 8.29 | 7.49 | 7.64 |
| 4 | 8.33 | 8.28 | 7.62 |
| 42 | 7.31 | 7.11 | 7.55 |
| 20 | 8.37 | 8.02 | 7.48 |
| 22 | 8.42 | 7.40 | 7.46 |
| 16 | 8.07 | 8.36 | 7.39 |
| 43 | 7.39 | 6.98 | 7.35 |
| 6 | 7.55 | 7.63 | 7.34 |
| 1 | 8.48 | 8.35 | 7.20 |
| 48 | 6.63 | 6.52 | 7.07 |
| 41 | 7.01 | 6.53 | 7.02 |
| 24 | 7.57 | 7.45 | 6.97 |

### Comparative Data

Table 4 below demonstrates that the affinity for the D₂ receptor is significantly greater for two compounds according to the invention in comparison with the closest analog disclosed in the above-mentioned WO publication WO 99/19317. The values in the Table are pIC₅₀ values and were determined in accordance with the procedure given above for determining D₂ affinity.

TABLE 4
Comparative in vitro data of two compounds according to the invention with a
corresponding prior art analogue

| Co. No. | h-D2_{L} | Structure |
|---|---|---|
| Prior art compound 21from WO 99/19317 | 6.96 | 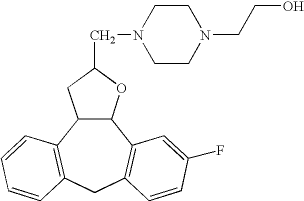 |
| 28 | 8.34 | 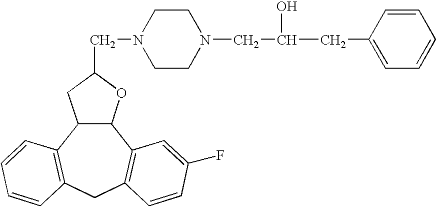 |
| 29 | 8.22 | 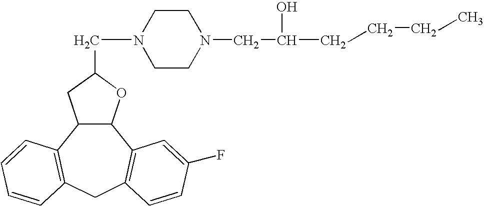 |

### D. Composition Examples

“Active ingredient” (A.I.) as used throughout these examples relates to a compound of Formula (I), a pharmaceutically acceptable acid or base addition salt thereof, a stereochemically isomeric form thereof, an N-oxide form thereof, and a quaternary ammonium salt thereof

### EXAMPLE D.1

### Oral Solution

Methyl 4-hydroxybenzoate (9 g) and propyl 4-hydroxybenzoate (1 g) were dissolved in boiling purified water (4 l). In 3 l of this solution were dissolved first 2,3-dihydroxybutanedioic acid (10 g) and thereafter A.I (20 g). The latter solution was combined with the remaining part of the former solution and 1,2,3-propanetriol (12 l) and sorbitol 70% solution (3 l) were added thereto. Sodium saccharin (40 g) were dissolved in water (500 ml) and raspberry (2 ml) and gooseberry essence (2 ml) were added. The latter solution was combined with the former, water was added q.s. to a volume of 20 l providing an oral solution comprising 5 mg of the active ingredient per teaspoonful (5 ml). The resulting solution was filled in suitable containers.

### EXAMPLE D.2

### Film-Coated Tablets

### Preparation of Tablet Core

A mixture of A.I. (100 g), lactose (570 g) and starch (200 g) was mixed well and thereafter humidified with a solution of sodium dodecyl sulfate (5 g) and polyvinylpyrrolidone (10 g) in water (200 ml). The wet powder mixture was sieved, dried and sieved again. Then there was added microcrystalline cellulose (100 g) and hydrogenated vegetable oil (15 g). The whole was mixed well and compressed into tablets, giving 10.000 tablets, each containing 10 mg of the active ingredient.

### Coating

To a solution of methyl cellulose (10 g) in denaturated ethanol (75 ml) there was added a solution of ethyl cellulose (5 g) in dichloromethane (150 ml). Then there were added dichloromethane (75 ml) and 1,2,3-propanetriol (2.5 ml). Polyethylene glycol (10 g) was molten and dissolved in dichloromethane (75 ml). The latter solution was added to the former and then there were added magnesium octadecanoate (2.5 g), polyvinylpyrrolidone (5 g) and concentrated color suspension (30 ml) and the whole was homogenated. The tablet cores were coated with the thus obtained mixture in a coating apparatus.

### EXAMPLE D.3

### Injectable Solution

Methyl 4-hydroxybenzoate (1.8 g) and propyl 4-hydroxybenzoate (0.2 g) were dissolved in boiling water (500 ml) for injection. After cooling to about 50° C. there were added while stirring lactic acid (4 g), propylene glycol (0.05 g) and A.I. (4 g). The solution was cooled to room temperature and supplemented with water for injection q.s. ad 1000 ml, giving a solution comprising 4 mg/ml of A.I. The solution was sterilized by filtration and filled in sterile containers.

1. Compound according to Formula (I): 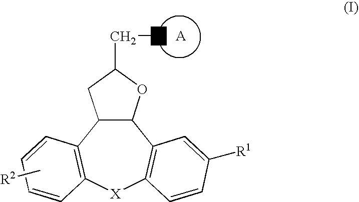
a pharmaceutically acceptable acid or base addition salt thereof, a stereochemically isomeric form thereof, an N-oxide form thereof, and a quaternary ammonium salt thereof, wherein:
R¹ is hydrogen, halo or C₁₋₆alkyloxy;
R² is hydrogen or cyano; and
a) X is O or S; and
A is a radical of formula (a-1), (a-2) or (a-3), 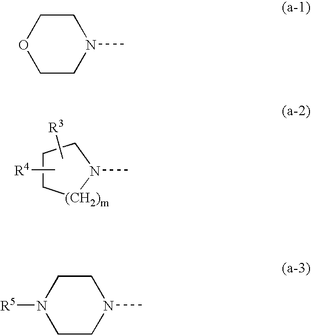
wherein:
m is an integer equal to zero, 1, 2 or 3;
R³ and R⁴ are each independently hydrogen, C₁₋₆alkyl or aryl; and
R⁵ is hydrogen; C₁₋₆alkyl; C₁₋₆alkylcarbonyl; C₁₋₆alkyl-carbonyloxyalkyl; C₁₋₆alkyloxycarbonyl; aryl; or C₁₋₆alkyl substituted with one or more substituents selected from hydroxy, C₁₋₆alkyloxy, C₁₋₆alkylcarbonyloxy and aryl; or
b) X is CH₂; and
A is a radical of formula (a-2) or (a-3) above wherein:
m is an integer equal to zero, 1, 2 or 3;
R³ and R⁴ are each independently hydrogen or C₁₋₆alkyl; and
R⁵ is hydrogen; C₂₋₆alkyl; C₁₋₆alkylcarbonyl; C₁₋₆alkylcarbonyloxyalkyl; C₁₋₆alkyloxycarbonyl; or C₁₋₆alkyl substituted with one or more substituents selected from hydroxy and aryl, with the proviso that 2-hydroxyethyl is excluded; and
aryl is phenyl; or phenyl substituted with 1, 2 or 3 substituents selected from halo, hydroxy, C₁₋₆alkyl and halomethyl.

2. Compound according to claim 1, wherein:
R¹ is halo;
R² is hydrogen;
aryl is phenyl; or phenyl substituted with halo or halomethyl.

3. Compound according to any one of claim 1 or 2, wherein:
X is O or S;
A is a radical of formula (a-1), (a-2) or (a-3) above, wherein
m is an integer equal to 1 or 2;
R³ and R⁴ are each independently hydrogen or aryl; and
R⁵ is C₁₋₆alkyl or C₁₋₆alkyl substituted with an hydroxy substituent.

4. Compound according to claim 1 wherein the compound has a structural formulas selected from the group consisting of:

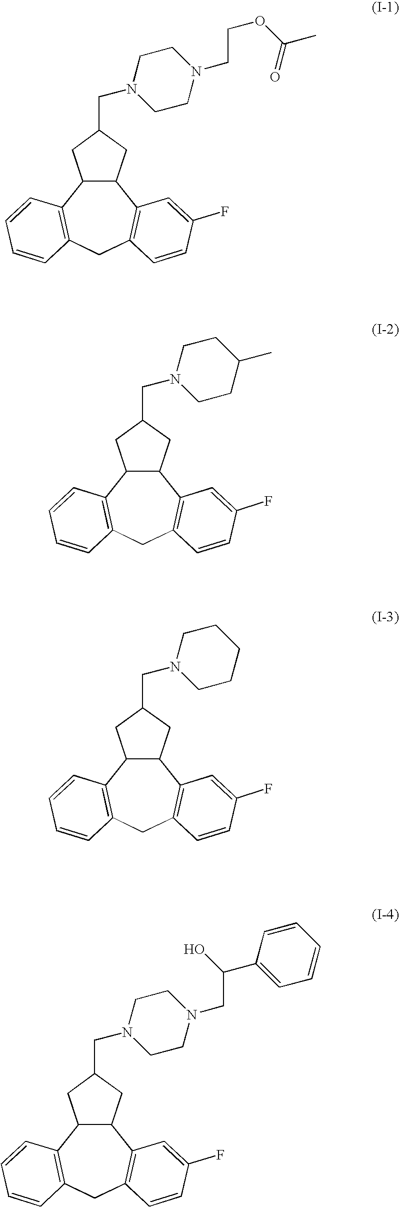

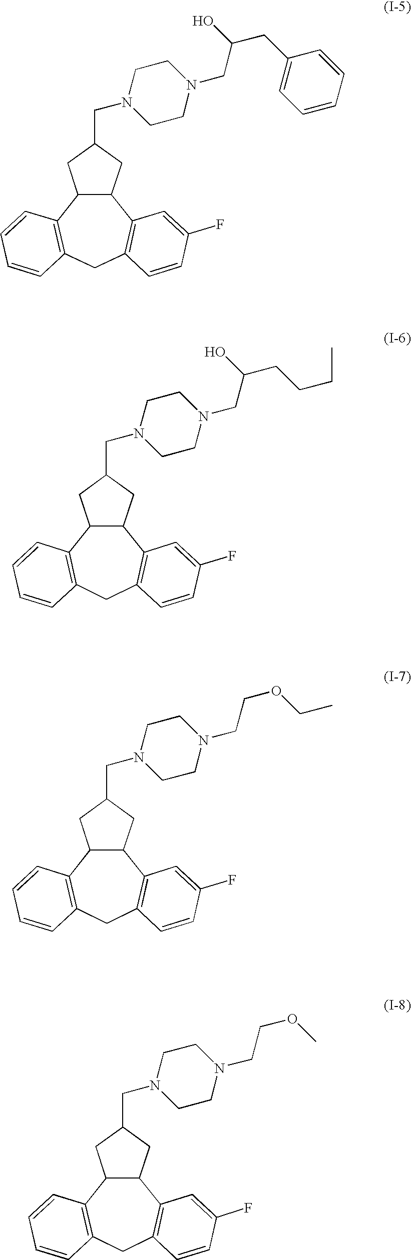

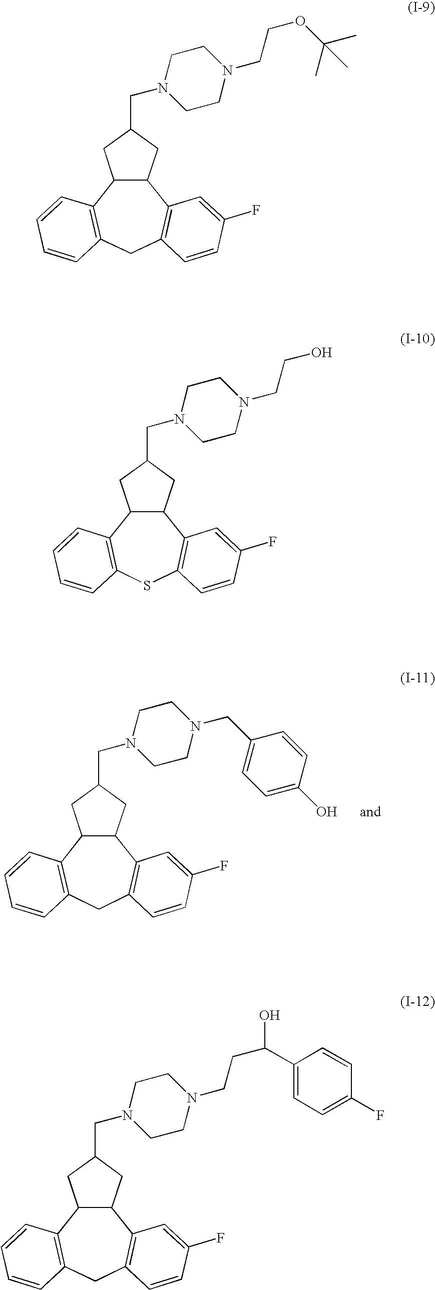

5. Compound of claim 1 4, wherein the salt is trifluoroacetate, oxalate, or mandelate.

6. Compound of claim 1 for use as a medicine.

7. A method of treating a patient in need of treatment for conditions, either prophylactic or therapeutic or both, mediated through the 5-HT₂ and/or D₂ receptor comprising administering a therapeutically effective amount of a compound of claim 1 to a patient in need of treatment.

8. The method of claim 7 wherein the conditions mediated through the 5-HT₂ and/or D₂ receptor is selected from the group consisting of anxiety, bipolar disorders, sleep-disorders, sexual disorders, psychosis, borderline psychosis, schizophrenia, migraine, personality disorders, obsessive-compulsive disorders, social phobias, panic attacks, organic mental disorders, mental disorders in children, aggression, memory disorders and attitude disorders in older people, addiction, obesity, bulimia and similar disorders.

9. The method of claim 8 wherein the conditions mediated through 5-HT₂ and/or D₂ receptor is selected from the group consisting of anxiety, psychosis, schizophrenia, migraine and addictive properties of drugs of abuse.

10. Pharmaceutical composition comprising a pharmaceutically acceptable carrier and, as active ingredient, a therapeutically effective amount of a compound according to claim 1.

11. Process for the preparation of a composition as claimed in claim 10, characterized in that a pharmaceutically acceptable carrier is intimately mixed with a therapeutically effective amount of a compound claim 1.

12. Process for the preparation of compounds of formula (I) which comprises N-alkylating an intermediate compound of formula (II) with an intermediate compound of formula (III) 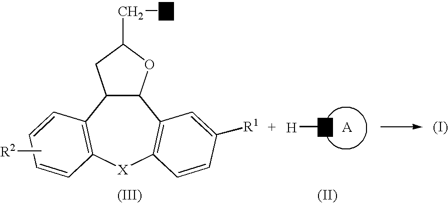
wherein R¹, R², X and the cyclic moiety A are as defined in claim 1 and W is a suitable leaving group, such as halo or an organosulfonyl group.
